(19) United States
(12) Patent Application Publication
(10) Pub. No.: US 2020/0281771 A1
(43) Pub. Date: Sep. 10, 2020

(54) Movement Aid for the Visually Impaired

(71) Applicants: SivaGouzia Sivarajah, Frisco, TX (US); SivaKrish Sivarajah, Frisco, TX (US); SivaKeirth Sivarajah, Frisco, TX (US)

(72) Inventors: SivaGouzia Sivarajah, Frisco, TX (US); SivaKrish Sivarajah, Frisco, TX (US); SivaKeirth Sivarajah, Frisco, TX (US)

(21) Appl. No.: 16/810,179

(22) Filed: Mar. 5, 2020

Related U.S. Application Data
(60) Provisional application No. 62/814,163, filed on Mar. 5, 2019.

(51) Int. Cl. A61F 9/08; G08B 7/06; G06K 9/00; G06K 9/62; G06K 9/46

(52) U.S. Cl. CPC A61F 9/08; G08B 7/06; G06K 9/00671; H04W 4/90; G06K 9/00288; G06K 9/4652; G06K 9/6256

(57) ABSTRACT

A movement aid for the visually impaired (MAVI) device, comprising a computer vision imaging device configured to capture one or more image streams on a path of a user, a processor coupled to the computer vision imaging device and configured to obtain vector image data from the image stream, wherein the vector image data comprises at a timestamp, positional coordinates, and directional information related to one or more objects identified in the vector image data, compare the vector image data with historical vector image data to detect an object within a proximity range of the MAVI device, and notify the user when an object is detected within the proximity range of the MAVI device.

### CROSS-REFERENCE TO RELATED APPLICATIONS

This patent application claims the benefit of U.S. Provisional Patent Application No. 62/814,163, filed Mar. 5, 2019 by SivaGouzia. Sivarajah, et al., and titled “Movement Aid for the Visually Impaired,” which is hereby incorporated by reference.

### BACKGROUND

Blind and visually impaired individuals mainly use a cane to travel and navigate, while a small percent of individuals use a guide dog. However, using a cane only provides feedback regarding objects on the ground-level. Thus, even with the cane, blind individuals may hit their heads and shoulders on obstacles above ground-level since the cane does not detect the object. Blind or visually impaired pets and animals cannot use walking aids such as a cane or walker. Therefore, these pets and animals struggle to walk and move around because other resources are not otherwise available to guide them. Similarly, patients such as those suffering from Alzheimer's disease or other similar diseases frequently bump into doors, surrounding objects, or moving objects.

In addition, several occupations require employees to move about environments with limited visibility. For example, firefighters and emergency workers intentionally move through hazardous environments surrounded by smoke or fire. In these environments, the firefighters and emergency workers may collide with objects that are moving and stationary while trying to respond to the emergency situation.

Further, automobiles often travel through extreme weather environments in which the driver of the automobile has impaired vision due to the extreme weather environment. For example, automobiles frequently travel through heavy rain, wind, and fog, which impairs the vision of the driver while driving the automobile.

### SUMMARY

In accordance with the various embodiments disclosed herein a movement aid for the visually impaired (MAVI) device (also referred to herein as a “MAVI system”) may increase the ability of blind and visually impaired individuals, pets, or animals. In some embodiments, the MAVI device may also assist patients suffering from diseases, distracted people, firefighters, and automobiles to move safely and efficiently. For example, the MAVI device assists Alzheimer's patients or patients suffering from other diseases to move safely, thus preventing accidents and injuries. The MAVI device also helps firefighters or policemen move through fog, smoke, or other hazardous environments safely when visibility is impaired. The MAVI device can also be used with automobiles to prevent collision with other moving and/or stationary objects. In some embodiments, the MAVI can detect objects in a larger range than a cane and can detect in multiple directions. For example, the MAVI device detects low hanging objects and objects to the side of a user. Additionally, the MAVI device comes with safety precautions which allow the user to send a current location to a friend or family member. Therefore, the MAVI device may provide object detection information to individuals to more quickly determine an object's location, and to provide more time to accommodate it. The MAVI device uses computer vision, artificial intelligence, machine learning, data analytics, sensors, mobile APP, etc. to detect objects with which a user wearing the MAVI device may collide.

In an embodiment, the disclosure includes a MAVI device comprising a computer vision imaging device configured to capture one or more image streams on a path of a user, a processor coupled to the computer vision imaging device and configured to obtain vector image data from the image stream, wherein the vector image data comprises at a timestamp, positional coordinates, and directional information related to one or more objects identified in the vector image data, compare the vector image data with historical vector image data to detect an object within a proximity range of the MAVI device, and notify the user when an object is detected within the proximity range of the MAVI device.

In another embodiment, the disclosure includes a method performed by a MAVI device comprising capturing, by a computer vision imaging device, an image stream on a path of a user, obtaining, by a processor coupled to the computer vision imaging device, vector image data from the image stream, wherein the vector image data comprises at least one of a timestamp, positional coordinates, or directional information related to one or more objects identified in the vector image data, comparing, by the processor, the vector image data with historical vector image data to detect an object within a proximity range of the MAVI device, and notifying, by the processor, the user when an object is detected within the proximity range of the MAVI device.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 illustrates a MAVI primary device in accordance with various embodiments;

FIG. 2 illustrates a MAVI secondary device in accordance with various embodiments;

FIG. 3 illustrates a side-profile of a user operating the MAVI system in accordance with various embodiments;

FIG. 4 illustrates a front-profile of a user operating the MAVI system in accordance with various embodiments;

FIG. 5 illustrates a schematic block of figures of a MAVI primary device in accordance with various embodiments;

FIG. 6 illustrates a user operating the MAVI system with a walking cane in accordance with various embodiments;

FIG. 7 illustrates a user operating the MAVI system with a guide animal in accordance with various embodiments;

FIG. 8 illustrates a user operating the MAVI system with a walker in accordance with various embodiments;

FIG. 9 illustrates a first responder operating the MAVI system in accordance with various embodiments;

FIG. 10 illustrates an animal operating the MAVI system in accordance with various embodiments; and

FIG. 11 illustrates a computer system in accordance with various embodiments.

### DETAILED DESCRIPTION

It should be understood at the outset that, although illustrative implementations of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

Disclosed herein are various embodiments related to one or more MAVI devices and various application scenarios. A MAVI device is a device that helps blind and visually impaired individuals travel safely with or without a cane or other assistance object, and aids in collision avoidance. The MAVI device can detect nearby objects in one or more directions.

In an embodiment, the MAVI device comprises a plurality of computer vision imaging devices, such as a camera. The computer vision imaging device is configured to capture a computer vision image stream in an area in front of the MAVI device on the path of the user. The computer vision image stream includes an image vector data stream. The image vector data stream comprises a plurality of image vectors for images captured by the computer vision imaging device over a predefined period of time and associated vector image data. The vector image data includes data describing the associated image and objects detected in the associate image. For example, the vector image data includes position coordinates, direction, GPS coordinates, and timestamp.

In an embodiment, Visual Processing Units (VPU) in the computer vision imaging device is configured to rapidly process the captured image stream in real-time, identify objects from each of the images in the image stream, and feed the data to the central processing unit (CPU) of the computer vision imaging device. The CPU uses the data to detect objects with which a user may collide and analyzes a speed of the object and a relative distance between the user and the object. The CPU uses the speed of the object and/or the relative distance between the user and the object to determine whether to notify the user of the MAVI device of the object.

In an embodiment, the vector image data for the image stream is stored locally at the MAVI device or stored remotely at a remote server. For example, the MAVI device may forward the vector image data for the image stream to the remote server using wireless modules (over 4G/LTE/5G or WiFi communication or BLUETOOTH modules.

In various embodiments, the MAVI device is configured to training a machine learning model to facilitate detecting objects in front of the MAVI device. In an embodiment, the MAVI device collects vector image data from one or more image streams and uses the collected data to train the MAVI device to automatically determine objects and notify the user of the objects. A trained MAVI device can understand a complex data set and recognize patterns within the data set to intelligently identify objects within a user's path and notify the user as to a possible collision with the object. The MAVI device collects and stores large amount of data that is clustered and analyzed to be used for training and object detection. The collected data is processed through complex calculations and algorithms to produce intelligent results. For example, a movement of an object may be determined and training by referencing timestamps of images, position coordinates of objects within the image, and/or the appearance of disappearance of objects within the image.

In various embodiments, the MAVI device comprises a computer vision imaging device that is configured to capture an image stream on a path of a user of the MAVI device. The MAVI device further comprises a processor coupled to the computer vision imaging device. The processor is configured to obtain vector image data from the image stream, and compare the vector image data with historical vector image data to identify an object within a proximity range of the MAVI device. In an embodiment, the historical vector image data comprises data describing environments and objects that are similar to a current environment of the user. The processor is also configured to notify the user when an object is detected within the proximity range of the MAVI device.

In various embodiments, the processor is further configured to collect vector image data for a plurality of image streams over a period of time to obtain the historical vector image data, and train a machine learning model based on the historical vector image data to recognize patterns in detecting objects on the path of the user. The more data that is collected, the more accurate the MAVI device becomes in detecting objects. In an embodiment, the MAVI device is configured to perform face recognition and color recognition on an image in the image stream. In this way, the MAVI device enables visually impaired users to be aware of the people and colors in an environment surrounding the user, for enhanced social interactions.

In an embodiment, the MAVI device comprises a selectable button, which, when selected, causes the MAVI device to contact an emergency responder. In an embodiment, the MAVI device uses a built-in wireless module to contact the emergency responder. In another embodiment, the MAVI device is configured to communicate coupled to the user's smartphone, such that the MAVI device instructs the user's smartphone to contact the emergency responder. If the MAVI device has a built-in wireless modem, the emergency contact button and remote communication button can be programmed using a web interface where the emergency responders contact information, other contact information, preset messages, etc. may be predefined. In an embodiment, the messages sent to remote contacts include the GPS coordinate location.

In some embodiments, the MAVI device comprises selectable button, which, when selected, enables at least one of an indoor mode, an outdoor mode, a crowded mode, or a non-crowded mode. The indoor mode and the outdoor mode may preset the MAVI device to be muted and to detect objects within a reduced proximity range, while the outdoor mode or the non-crowded mode may preset the MAVI device to be louder and detected objects within an increased proximity range.

In some embodiments, the MAVI device uses smart phone and smart devices to collect and/or send data. The user of the MAVI device may select features to be used for training the MAVI device and/or detecting objects within a certain vicinity. For example, a user of the MAVI device may also be a smartphone user who prefers to use smartphone GPS and wireless communication. In an embodiment, the MAVI device may be communicatively coupled to a user's smartphone, and in this way, the MAVI device can use the BLUETOOTH and GPS features of the mobile device. The MAVI device may also have access to the calendar, contacts, messages, and other data of the user's smartphone.

In some embodiments, the MAVI device can detect stationary objects or moving objects. When detecting stationary objects, the MAVI device compares the distance between the MAVI device and the object being detected to determine whether a difference between the distance and a base point is greater than a threshold. If the difference between the distance and the base point is greater than the threshold, the MAVI device notifies the user of a possible collision with an object.

When detecting moving objects, the MAVI device uses an “n” seconds rule to detect objects and avoid collisions. In some embodiments, the MAVI device determines a velocity and/or acceleration of the moving object over “n” seconds to determine whether the user of the MAVI device is likely to collide with the moving object. When the MAVI device detects that a collision with the moving object is likely, the MAVI device notifies the user of the moving object.

There are several situations in which the MAVI device notifies the user of a potential collision with a detected object. In one case, the user may not be moving, but the detected object may be moving toward to the user. For example, the user may not be moving, but an object, such as a car or a moving ball, may be moving towards the user. In an embodiment, the MAVI device calculates a probability of colliding with the detected object, and notifies the user of the detected object based on the calculated probability. In a second case, the user may be moving a speed of X m/s, and a stationary object may be in the path of the user. For example, the user may be walking normally, and a door, wall, or other object may be present on the user's path. In an embodiment, the MAVI device detects the object and notifies the user of the object being on the user's path. In a third case, both the user and the object are moving. For example, two visually impaired people may be walking toward one another, or one visually impaired person may be walking toward a moving car. In an embodiment, the MAV device detects the moving object and determines a probability of colliding with the moving object. In this embodiment, the MAVI device notifies the user of the moving object based on the calculated probability, similar to the first case.

In various embodiments, the MAVI device determines a relative velocity, relative acceleration, and/or other factors to perform object detection, range, and notification. In an embodiment, the detection range increases as the speed of the moving object increases. The MAVI device uses one or more different types of technology, including, but not limited to, Visual Processing Units (VPU), Artificial Intelligent (AI), Machine Learning (ML), PIXY, facial recognition, facial detection, sensor technology, wireless, WiFi, BLUETOOTH, cloud services, Mobile App, third party applications, etc.

In some cases, a person's walking speed may vary greatly depending on many factors. The average human walking speed at crosswalks is about 1.4 meters per second (m/s). Human reaction time may be affected by various factors, such as age, fatigue, disease, illness, or even medications. The average human may observe/react to an object in a tenth of a second to four tenths of a second. A user operating an automobile observes similar reaction times. Animals have a large variance of reaction times. The MAVI device takes these factors into account when determining whether a user should be notified of a possible collision of an object.

For example, a MAVI device may detect objects 2 feet (ft) to the side of a person, anything on the ground (i.e., steps, ditches, curbs), any low hanging objects, and objects up to 10 ft in front of the individual. The MAVI device may be configured to change the distance that it can detect in the front from, for example, 10 ft to 5 ft with the push of a button. Additionally, the MAVI device may have a safety feature which allows the blind individual to send their current location to friend or family member with the single push of a button. In some embodiments, the MAVI device also uses cameras to detect colors, objects, and facial features.

In various embodiments, the MAVI device uses one or more forms of notifications to notify the user of an object, such as a vibration, an audio signal, or a speech dictation identifying the object detected. The audio signal can include audio ringtone or chime, voice, beeping, or buzzing. The MAVI device may also initiate the video camera, contact a remote system/user, or contact an emergency responder in response to a user input from a user of the MAVI device. A quantity or strength of the vibrations may indicate the relative distance, position, and/or importance of an object and/or collision probability. Further, notifications such as audio, voice, beeping, or buzzing may be used to indicate system properties, object properties, user guidance, or communications. In an embodiment, the MAVI device may send streaming videos to a remote user to assist the user of the MAVI device. Further, the video camera may be used to capture objects, landmarks, or an environment surrounding the user. For example, the environment may include weather, such as rain or lightening, or a surrounding, such as an oily floor. A remote user may obtain the streaming video, Global Positioning System (GPS) coordinates, object properties, and/or system properties to assist the user. The collected data may be analyzed locally, remotely, and/or in a cloud computing environment.

In some embodiments, the MAVI device is configured to detect when the user is moving away from his or her regular or routine path. For example, the MAVI device stores information regarding historical paths traveled by the user of the MAVI device, and the MAVI device is configured to determine regular or routine paths traveled by the user. In this case, the MAVI device is configured to determine when the user deviates from the regular or routine paths traveled by the user. In an embodiment, the MAVI device uses a smart phone or other smart devices to interact with the user and or emergency responders. In an embodiment, the MAVI uses an application for a web-server to share information and interact with users of the MAVI device.

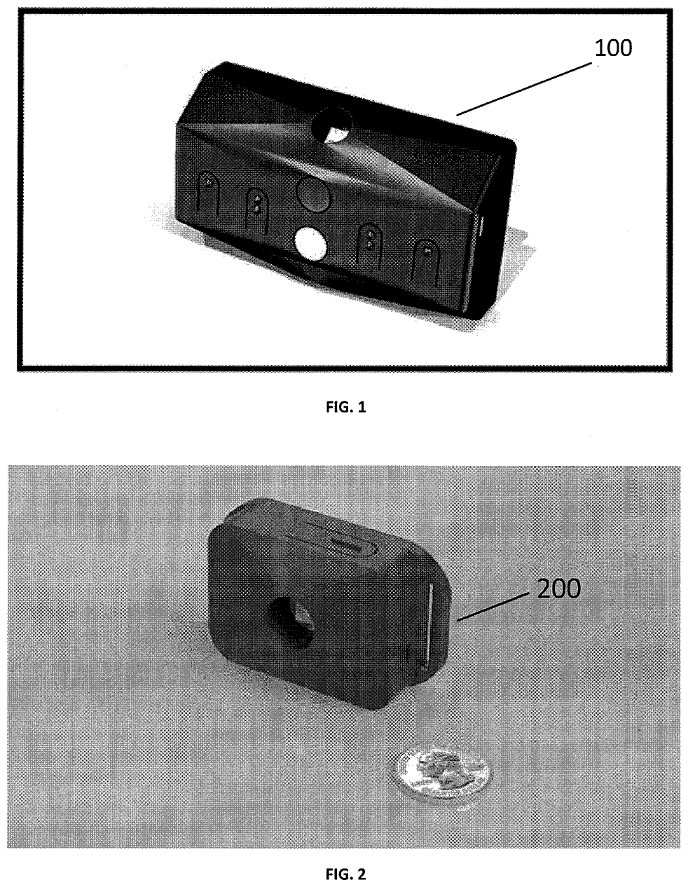

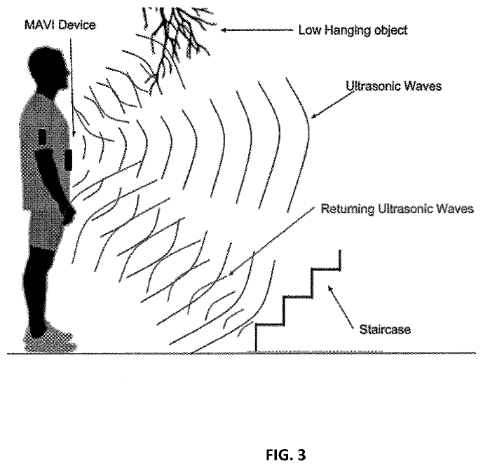

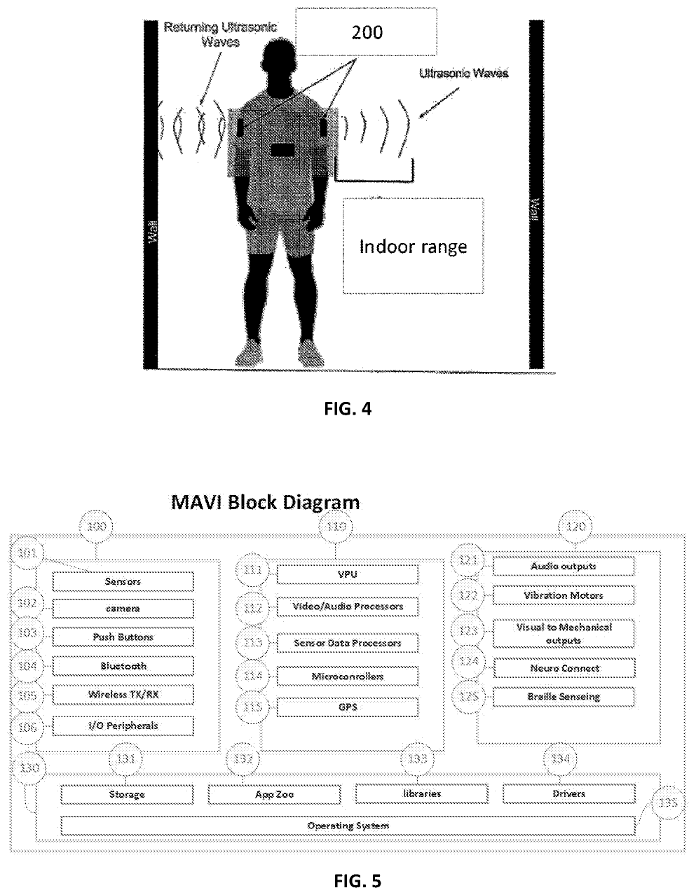

A MAVI system (also referred to herein as “MAVI device”) can include an individual wearing one or more MAVI devices. FIG. 1 shows an exemplary MAVI primary device 100. FIG. 2 shows an exemplary MAVI secondary device 200. As illustrated in FIGS. 3-4, a MAVI primary device 100 can be attached to a person's chest, waistline, or the belt. In an embodiment, the MAVI primary device 100 and/or the MAVI secondary device 200 comprises a clasp, which is detachably attachable to a user, cane, walker, automobile, or any other item. For example, the clasp may be buckle or a clamp. In some embodiments, the MAVI primary device 100 and the MAVI secondary device 200 are wearable devices. The MAVI primary device 100 detects anything around the head level of a person, anything on the ground, and anything in between. A MAVI secondary device 200 can be attached to a person's arm.

For both the MAVI primary device 100 and the MAVI secondary device 200, each device has an on/off button. If the MAVI devices 100,200 are turned on or off, a voice recording will alert the user via a speaker. A voice notification can also alert the user to the battery level as soon as the MAVI device(s) is turned on. In some embodiments, each MAVI device may announce a battery level at various thresholds, such as when the battery reaches 75%, 50%, 25%, 10%, 5%, and the like. Each MAVI device comprises a power source, a controller/processor, and can operate independent of other devices. Each MAVI device also has a sleep function, where if the distance between the device and the object doesn't change for a predetermined period of time (e.g., 5-10 seconds), then the respective MAVI device will go to into a sleep mode until there is a change in the detected distance.

Both the MAVI primary device 100 and the MAVI secondary device 200 can detect proximate objects around the blind individual using proximity sensors. In various embodiments, the MAVI device proximity sensor may be an ultrasonic sensor, an optical sensor, a radar sensor, or a combination thereof. The MAVI primary device 100 is configured to detect any low hanging object, anything on the ground, and anything up to 10 ft. in front of the blind individual. When the MAVI primary device 100 is detecting moving objects, the “n” seconds method may be used. For example, the MAVI primary device determines a relative velocity of a device as 1.5 m/s using the 2 second rule (detecting an object moving with a relative velocity of 1.5 m/s, applying the 2 second rule, then determining that the distance range is 1.5 m/s*2 second, which is 3 meters). The MAVI secondary device 200 is configured to detect anything 2 ft to the side of the individual. The provided distances of 10 ft and 2 ft are merely examples and are not limiting. The range of each device may be more or less than the provided examples, as would be understood by one skilled in the art. Further, in various embodiments, the proximity range of a MAVI device is adjustable and can be modified based on user input, such as with a dial or button. When the MAVI device sensor detects an object within the set proximity range, the MAVI device vibrates to alert the user. A vibrating motor or similar device can be used to generate the vibrations. In one embodiment, the vibration intensity is proportional to the proximity of the detected object(s). If the object is closer, then the intensity of the vibration is higher. If the object is far away, the intensity of the vibration is low.

The embodiments of the present disclosure may be used for many applications. First, the MAVI device is a feedback system that can be used for collision detection and prevention, remote assistance, user environment visibility or awareness, user alert and assistance, safety, and precaution. Blind and visually impaired people can use the MAVI device to travel safely. Blind and visually impaired pets or animals can use the MAVI device to prevent collisions. Firefighters can use the MAVI device in smoke filled or poor visibility environments. A person reading on a handheld device may use the MAVI device to prevent accidents or collision when not focusing on the path in front of him or her. Automobiles may also use the MAVI device to prevent collisions. The MAVI device can locate stationary objects on or next to a moving object path such as traffic lights or traffic situations.

In accordance with various embodiments and with reference to FIG. 5, the MAVI primary device may comprise inputs 100, processors 110, outputs 120, and common blocks 130. The inputs 100 include input devices, such as the sensors 101, camera 102, push buttons 103, BLUETOOTH receiver 104, wireless transmitters/receivers (TX/RX) 105, or I/O peripherals 106. The processors 100 include processing devices, such as the VPU 111, video/audio processors 112, sensor data processors 113, microcontrollers 114, or a GPS 115. The outputs 120 include output devices, such as audio outputs 121, vibration motors 122, visual-to-mechanical outputs 123, neuro connect outputs 124, or braille sensing outputs 125. The common blocks 130 include other computing devices, such as the storage 131, the feature interaction and configuration application zoo 132, mathematical, algorithm and code libraries 133, peripheral drivers 134, and operating systems 135.

The storage 131 may be, for example, a memory storing instructions to perform the embodiments disclosed herein. The storage 131 may be stored locally at the MAVI device or on a remote cloud sever. One of the processors 110 executes the instructions stored in the memory to perform the embodiments disclosed herein. One or more type of sensors 101 scan consistently collect various data, such as image streams in real-time. The plurality of sensors may include proximity sensors, gas and pollution detectors, infrared sensors, motion sensors, etc.

The camera 102 is a computer vision imaging device configured to capture streaming image data processed by a visual processing unit 111 or microcontrollers 113 to send to a local, remote, and/or cloud storage 131 using wireless TX/RX 105 or BLUETOOTH receiver 104 via a smart phone.

Further, the MAVI primary device can comprise a transceiver, such as a BLUETOOTH receiver 104. The attached system peripherals 106 provide surrounding environmental, social and remote information, process complex algorithms, perform mission critical tasks of object detection, facial and color recognition, computer vision sensing, neural network interactions, convert images and videos streaming to text, audio and mechanical functions, and social and environmental connectivity. For example, the VPU 111 performs computer vision sensing, the microcontroller converts images and videos streaming to text, and the application zoo 132 facilitates in social and environmental connectivity.

In various embodiments, the MAVI primary device can also include a location feature. A user can send a current location to a second device (such as a friend or family member's mobile phone) using a push button 113 or the like. In one embodiment, after receiving a user selection, the MAVI primary device can instruct the user's mobile device to obtain and send current GPS 115 of the user to a friend or family member. The MAVI primary device can be connected to the user's mobile device using a wired or wireless connection.

In various embodiments, the MAVI primary device or the MAVI secondary device also includes an emergency response feature. The MAVI primary device or the MAVI secondary device may comprise a selectable button, which, when selected, automatically contacts an emergency responder (e.g., 911) for immediate assistance.

In various embodiments, the MAVI primary device can comprise a camera. For example, the MAVI primary device may include a computer vision smart vision sensor. In other embodiments, the camera is separate from the MAVI primary device and they are wirelessly coupled to each other, such as via BLUETOOTH receiver 104 or wireless TX/RX 105. The camera can be configured to capture images of objects on an ongoing basis and provide the captured images to the controller for processing 111. The controller can perform image recognition and compare to a database of stored images 131. For example, the MAVI devices can perform facial detection and recognition (preprogrammed familiar faces); identify doors, elevators, outlets, bus stops, etc. Once an object is identified, the user can be notified through a speaker on the MAVI device.

An exemplary method of operation of the MAVI system on a user will now be described. Although specific values for distance and angles may described, these values are merely for illustrative purposes and are not limiting. In accordance with various embodiments and with renewed reference to FIGS. 1 and 3-4, the MAVI primary device 100 can comprise three proximity sensors. A first proximity sensor can be configured to detect objects at a 45° angle facing upwards and be set to measure anything at a 4 ft distance. The first proximity sensor detects objects at or above head level, and can alert the user via a sound (e.g., audio output 121), mechanical outputs 123, or braille sensing outputs 125. Neuro connect 124 is an advanced information feeding system which connects to eye nerve systems to capture camera streaming images. The eye nerves may connected via thin fiberoptic light signals. The second proximity sensor can be configured to detect objects at a 45° angle facing downward. The third proximity sensor can be configured to detect objects in the coverage area between the coverage areas of the first and second proximity sensors.

The second proximity sensor can be used to detect objects on the ground, bumps, ditches, etc. In various embodiments, the second proximity sensor includes a learning phase function. If a push button is pressed then the bottom ultrasonic sensor will measure the distance from itself to the ground for a period of 5 seconds. It will then calculate the average distance from itself to the ground and that will be its base point. This ultrasonic sensor will be connected with a speaker, and the speaker will play a tune if the average distance from the ground changes by 1 inch. If the average distance from the ground increases by “X” inches then that means there is a significant drop-off in the path of the person and a tune will be played. If the average distance from the ground decreases by “Y” inches then that means there is a bump in the path of the person and a different tune will be played. The “X” and “Y” inches threshold can be either preprogrammed or set by the user.

In various embodiments, the third proximity sensor is placed in the middle of the MAVI primary device 100. This sensor detects objects in between the first and second proximity sensors. In one embodiment, the third proximity sensor has a range of 10 ft, and the range can be modified (e.g., to 5 ft) using a button. In various embodiments, the third proximity sensor is connected with a vibration motor. If the third proximity sensor picks up any object then the vibration motor will vibrate. The closer the object the more intense the vibration will be.

In various embodiments, the MAVI primary device 100 can comprise four proximity sensors. A first proximity sensor of the four proximity sensors is positioned in parallel with a second proximity sensor of the four proximity sensors in a first direction. A third proximity sensor and a fourth proximity sensor of the four proximity sensors are positioned in between the first proximity sensor and the second proximity sensor. The third proximity sensor is positioned in parallel with the fourth proximity sensor in a second direction.

The first proximity sensor can be configured to detect objects at a predefined angle facing upwards and set to measure anything within a predefined distance, and the second proximity sensor can be configured to detect objects at a predefined angle facing downwards and set to measure anything within a predefined distance. The third proximity sensor can be configured to detect objects at a predefined angle facing toward the right and set to measure anything within a predefined distance, and the fourth proximity sensor can be configured to detect objects at a predefined angle facing toward the left and set to measure anything within a predefined distance. For example, each of the predefined angles may be 45°.

Additional advantages gained with the MAVI system include that it is not obvious to surrounding people that the user is visually impaired. This is an important benefit if a legally blind individual does not want to be seen as blind and does not use a cane. Also, the MAVI system gives the user a larger detection range than a cane, thereby improving its efficiency.

Although described above with respect to a user wearing the MAVI system, other applications and embodiments are also possible. The MAVI system is a wearable or attachable technology device that guides visually impaired people or animals, people with difficulty detecting surroundings, Alzheimer patients, firefighters or any person who have no visibility to navigate easily, stay connected with their surroundings, and have environmental and social awareness. A user can wear one or more devices on himself/herself, or on objects or animals that they use to navigate or get responses. For example, the MAVI device may be coupled to a cane, walker, or wheelchair. In various other embodiments, the MAVI device can include additional sensors to track/provide additional information related to the user, such as heart rate, body temperature and/or environmental temperature, and the like.

The MAVI devices in the following embodiments can be either the MAVI primary device 100 or the MAVI secondary device 200 as previously described. The specific features and functionality is the same and will not be repeated herein.

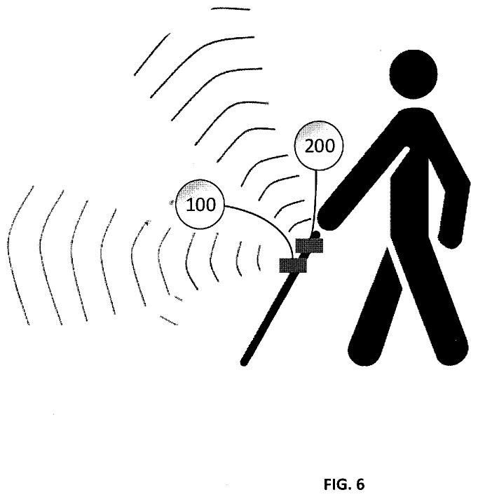

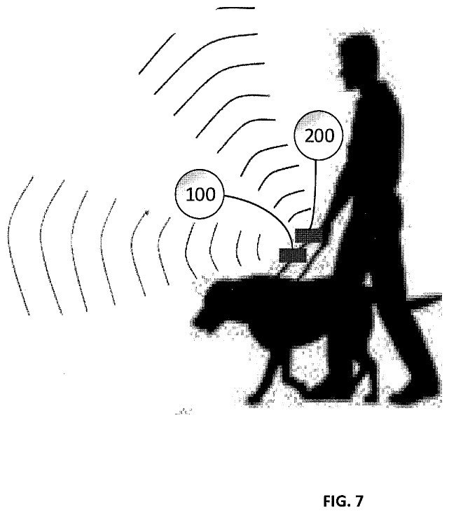

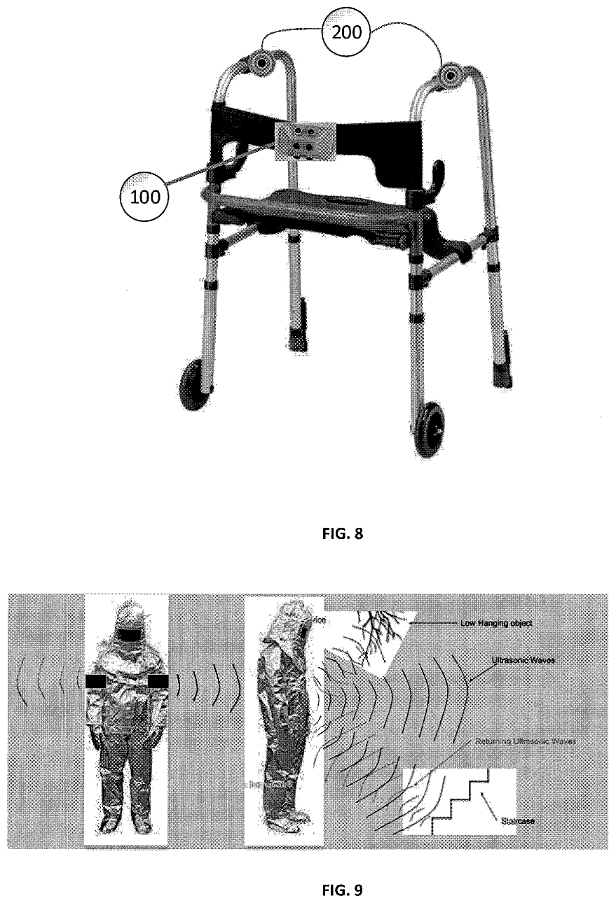

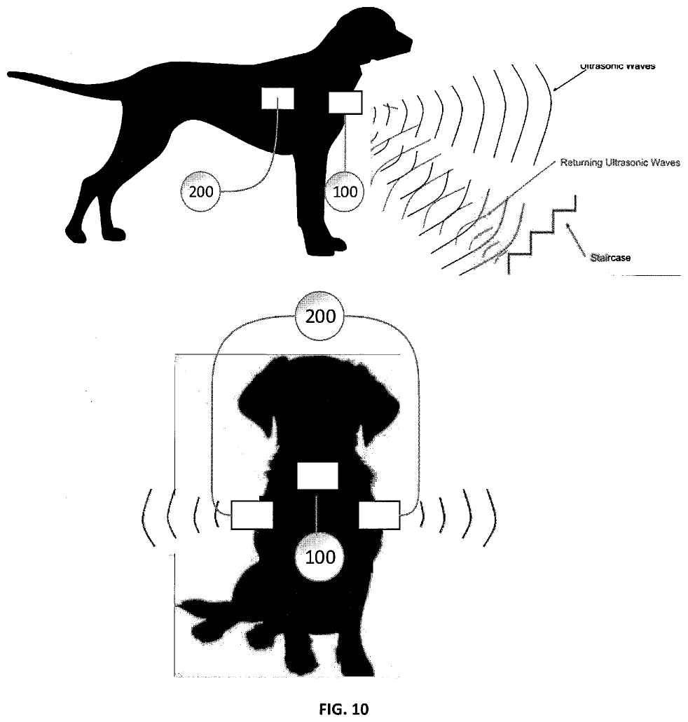

With reference to FIG. 6, the MAVI system can be used with a walking cane. Two MAVI devices can be attached to the cane, with one of the MAVI devices facing up and the other facing forward. The devices can be attached with a special molding design. FIG. 7 illustrates a similar application using the MAVI system attached to a leash of a guide dog. FIG. 8 illustrates a MAVI primary device 100 attached to a walker to assist visually impaired users with navigating. One or more MAVI secondary devices 200 can also be attached to aid in detecting objects on the side. In addition, the MAVI system can be used by emergency personal, firefighters, first responders, and the like to allow them to navigate through hazardous areas, such as thick smoke as shown in FIG. 9. Furthermore, the MAVI system can also be attached to visually-impaired animals as shown in FIG. 10.

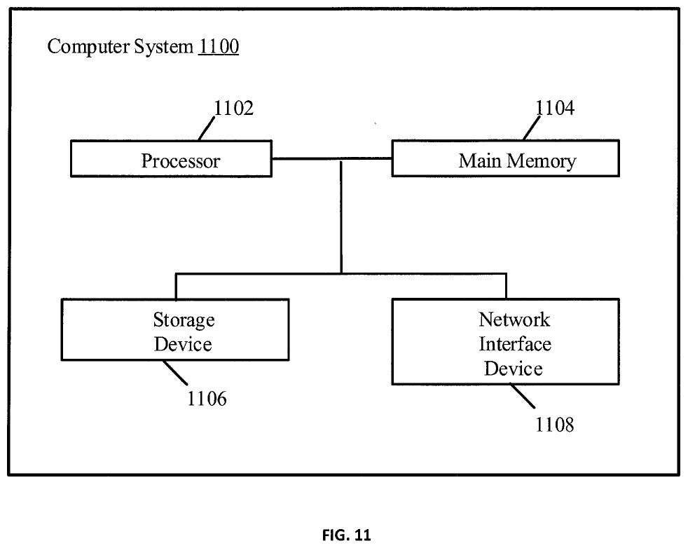

FIG. 11 shows a computer system 1100, which is illustrative of a computer system that may be used in connection with the various embodiments disclosed herein. The controller of the MAVI primary device 100 or the controller of the MAVI secondary device 200 may be implemented in the computer system 1100. The computer system 1100 may be illustrative of, for example, a laptop, a desktop computer, a computer within a node of several computers, or any other computing system that may be connected to a network of computers. The computer system 1100 comprises a processor 1102, and a main memory 1104 coupled to processor 1102. Additionally, processor 1102 and main memory 1104 may be coupled to storage device 1106, and a network interface device 1108.

Programs executable by the processor 1102 may be stored on the storage device 1106 (e.g., a hard drive, solid state disk, memory stick, optical disc), and accessed when needed by the processor 1102. Programs stored on the storage device 1106 may comprise programs to implement various processes on the computer system 1100. In some cases, the programs are copied from the storage device 1106 to the main memory 1104, and the programs are executed from the main memory 1104. Thus, both the main memory 1104 and storage device 1106 shall be considered computer-readable storage mediums.

In various embodiments, network interface device 1108 may allow computer system 1100 to exchange data over a wireless or wired network. In some embodiments the computer system 1100 may be connected to a plurality of other computers within a shared network. Thus, while many aspects may be performed serially, various embodiments enable parallel processing to speed up the overall processing time.

From the description provided herein, those skilled in the art are readily able to combine software with appropriate general-purpose or special-purpose computer hardware to create a computer system and/or computer subcomponents in accordance with the various embodiments and methods.

While several embodiments have been provided in the present disclosure, it may be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

Persons of ordinary skill in the art may understand that all or some of the steps of the methods in the embodiments may be implemented by a program instructing relevant hardware (such as a processor). The program may be stored in a computer readable storage medium. The storage medium may include a read-only memory (ROM), a random access memory (RAM), a magnetic disk, or an optical disc.

As used here, “at least one of,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” means A alone, B alone, C alone, A and B together, A and C together, B and C together, or A, C, and C together.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and may be made without departing from the spirit and scope disclosed herein.

What is claimed is:

1. A movement aid for the visually impaired (MAVI) device, comprising:
a computer vision imaging device configured to capture one or more image streams on a path of a user;
a processor coupled to the computer vision imaging device and configured to:
obtain vector image data from the image stream, wherein the vector image data comprises a timestamp, positional coordinates, and directional information related to one or more objects identified in the vector image data;
compare the vector image data with historical vector image data to detect an object within a proximity range of the MAVI device; and
notify the user when the object is detected within the proximity range of the MAVI device.

2. The MAVI device of claim 1, wherein the processor is further configured to:
collect vector image data for a plurality of image streams over a period of time to obtain the historical vector image data; and
train a machine learning model based on the historical vector image data to recognize patterns in detecting objects on the path of the user.

3. The MAVI device of claim 1, wherein the processor is further configured to perform face recognition on an image in the image stream.

4. The MAVI device of claim 1, wherein the processor is further configured to perform color recognition on an image in the image stream.

5. The MAVI device of claim 1, further comprises a selectable button, which, when selected, causes the MAVI device to contact an emergency responder.

6. The MAVI device of claim 1, further comprises a selectable button, which, when selected, enables at least one of an indoor mode, an outdoor mode, a crowded mode, or a non-crowded mode.

7. The MAVI device of claim 1, further comprising plurality of proximity sensors positioned on a first side of the MAVI device, wherein each of the proximity sensors is angled in a different direction.

8. The MAVI device of claim 7, wherein the plurality of proximity sensors comprises four proximity sensors, a first proximity sensor of the four proximity sensors being positioned in parallel with a proximity second sensor of the four proximity sensors in a first direction, wherein a third proximity sensor and a fourth proximity sensor are positioned in between the first proximity sensor and the second proximity sensor, and wherein the third proximity sensor is positioned in parallel with the fourth proximity sensor in a second direction.

9. The MAVI device of claim 7, wherein a first proximity sensor of the MAVI primary device is angled at 45 degrees (°) upward, wherein a proximity second sensor of the MAVI primary device is angled straight forward, and wherein a proximity third sensor of the MAVI primary device is angled at 45° downward.

10. The MAVI device of claim 1, wherein the user is notified of the object by at least one of a vibration, an audio signal, or speech dictation identifying the object.

11. A method performed by a movement aid for the visually impaired (MAVI) device, comprising:
capturing, by a computer vision imaging device, an image stream on a path of a user;
obtaining, by a processor coupled to the computer vision imaging device, vector image data from the image stream, wherein the vector image data comprises a timestamp, positional coordinates, and directional information related to one or more objects identified in the vector image data;
comparing, by the processor, the vector image data with historical vector image data to detect an object within a proximity range of the MAVI device; and
notifying, by the processor, the user when the object is detected within the proximity range of the MAVI device.

12. The method of claim 11, further comprising:
collecting, by the processor, vector image data for a plurality of image streams over a period of time to obtain the historical vector image data; and
training, by the processor, a machine learning model based on the historical vector image data to recognize patterns in detecting objects on the path of the user.

13. The method of claim 11, further comprising performing, by the processor, face recognition on an image in the image stream.

14. The method of claim 11, further comprising performing, by the processor, color recognition on an image in the image stream.

15. The method of claim 11, further comprising receiving, by a selectable button of the MAVI device, a selection causing the MAVI device to contact an emergency responder.

16. The method of claim 11, receiving, by a selectable button of the MAVI device, a user input enabling at least one of an indoor mode, an outdoor mode, a crowded mode, or a non-crowded mode.

17. The method of claim 11, wherein the MAVI device comprises a plurality of proximity sensors positioned on a first side of the MAVI device, wherein each of the proximity sensors is angled in a different direction.

18. The method of claim 17, wherein the plurality of proximity sensors comprises four proximity sensors, a first proximity sensor of the four proximity sensors being positioned in parallel with a proximity second sensor of the four proximity sensors in a first direction, wherein a third proximity sensor and a fourth proximity sensor are positioned in between the first proximity sensor and the second proximity sensor, and wherein the third proximity sensor is positioned in parallel with the fourth proximity sensor in a second direction.

19. The method of claim 17, wherein a first proximity sensor of the MAVI primary device is angled at 45 degrees (°) upward, wherein a proximity second sensor of the MAVI primary device is angled straight forward, and wherein a proximity third sensor of the MAVI primary device is angled at 45° downward.

20. The method of claim 11, wherein the user is notified of the object by at least one of a vibration, an audio signal, or speech dictation identifying the object.
